(19) United States
(12) Patent Application Publication
(10) Pub. No.: US 2014/0288169 A1
(43) Pub. Date: Sep. 25, 2014

(54) NICOTINIC RECEPTOR NON-COMPETITIVE ANTAGONISTS

(71) Applicants: Srinivasa Rao Akireddy, Winston-Salem, NC (US); Scott R. Breining, Winston-Salem, NC (US); Matt S. Melvin, Winston-Salem, NC (US); Srinivasa V. Murthy, Lewisville, NC (US); Anatoly A. Mazurov, Greensboro, NC (US); Balwinder Singh Bhatti, Winston-Salem, NC (US); Jon-Paul Strachan, Burlington, NC (US); Ronald Joseph Heemstra, Lewisville, NC (US); Todd Showalter, Clemmons, NC (US); Yunde Xiao, Clemmons, NC (US); Philip S. Hammond, Pinnacle, NC (US); Lan Miao, Advance, NC (US); David Kombo, Winston-Salem, NC (US); Daniel Yohannes, Winston-Salem, NC (US); Jason Speake, Winston-Salem, NC (US)

(72) Inventors: Srinivasa Rao Akireddy, Winston-Salem, NC (US); Scott R. Breining, Winston-Salem, NC (US); Matt S. Melvin, Winston-Salem, NC (US); Srinivasa V. Murthy, Lewisville, NC (US); Anatoly A. Mazurov, Greensboro, NC (US); Balwinder Singh Bhatti, Winston-Salem, NC (US); Jon-Paul Strachan, Burlington, NC (US); Ronald Joseph Heemstra, Lewisville, NC (US); Todd Showalter, Clemmons, NC (US); Yunde Xiao, Clemmons, NC (US); Philip S. Hammond, Pinnacle, NC (US); Lan Miao, Advance, NC (US); David Kombo, Winston-Salem, NC (US); Daniel Yohannes, Winston-Salem, NC (US); Jason Speake, Winston-Salem, NC (US)

(73) Assignee: TARGACEPT, INC., Winston-Salem, NC (US)

(21) Appl. No.: 13/978,583

(22) PCT Filed: Jan. 5, 2012
(86) PCT No.: PCT/US12/20246

§ 371 Date: Sep. 27, 2013

Related U.S. Application Data
(60) Provisional application No. 61/430,640, filed on Jan. 7, 2011.

(51) Int. Cl. C07D 493/08; C07C 211/38

(52) U.S. Cl. CPC C07D 493/08; C07C 211/38
USPC 514/470; 564/460; 514/661; 549/463

(57) ABSTRACT

The present invention relates to compounds that modulate nicotinic receptors as non-competitive antagonists, methods for their synthesis, methods for their use, and their pharmaceutical compositions.

### FIELD OF THE INVENTION

The present invention relates to compounds that modulate nicotinic receptors as non-competitive antagonists, methods for their synthesis, methods for their use, and their pharmaceutical compositions.

### BACKGROUND OF THE INVENTION

Nicotinic receptors are targets for a great number of exogenous and endogenous compounds that allosterically modulate their function. See, Arias, H. R., Binding sites for exogenous and endogenous non-competitive inhibitors of the nicotinic acetylcholine receptor, Biochimica et Biophysica Acta—Reviews on Biomembranes 1376: 173-220 (1998) and Arias, H. R., Bhumireddy, P., Anesthetics as chemical tools to study the structure and function of nicotinic acetylcholine receptors, Current Protein & Peptide Science 6: 451-472 (2005). The function of nicotinic receptors can be decreased or blocked by structurally different compounds called non-competitive antagonists (reviewed by Arias, H. R., Bhumireddy, P., Bouzat, C., Molecular mechanisms and binding site locations for noncompetitive antagonists of nicotinic acetylcholine receptors. The International Journal of Biochemistry & Cell Biology 38: 1254-1276 (2006)).

Non-competitive antagonists comprise a wide range of structurally different compounds that inhibit receptor function by acting at a site or sites different from the agonist, or orthosteric, binding site. Receptor modulation has proved to be highly complex for most non-competitive antagonists. The mechanisms of action and binding affinities of non-competitive antagonists differ among nicotinic receptor subtypes (Arias et al., 2006). Non-competitive antagonists may act by at least two different mechanisms: an allosteric and/or steric mechanism.

Allosteric mechanisms involve the binding of non-competitive antagonists to the receptor and stabilization of a non-conducting conformational state, namely, a resting or desensitized state, and/or an increase in the receptor desensitization rate. In contrast, the steric mechanism of antagonism is typically conceived as physical blockage (blockade) on the ion channel by the antagonist molecule. Antagonists of this latter type are termed non-competitive channel blockers (NCBs). Some inhibit the receptors by binding within the pore when the receptor is in the open state, thereby physically blocking ion permeation. While some act only as pure open-channel blockers, others block both open and closed channels. Such antagonists inhibit ion flux through a mechanism that does not involve binding at the orthosteric sites, and such inhibitions (blockade) can occur to varying degrees.

Barbiturates, dissociative anesthetics, antidepressants, and certain steroids have been shown to inhibit nicotinic receptors by allosteric mechanisms, including open and closed channel blockade. Studies of barbiturates support a model whereby binding occurs to both open and closed states of the receptors, resulting in blockade of the flow of ions. See, Dilger, J. P., Boguslaysky, R., Barann, M., Katz, T., Vidal, A. M., Mechanisms of barbiturate inhibition of acetylcholine receptor channels, Journal General Physiology 109: 401-414 (1997). Although the inhibitory action of local anesthetics on nerve conduction is primarily mediated by blocking voltage-gated sodium channels, nicotinic receptors are also targets of local anesthetics. See, Arias, H. R., Role of local anesthetics on both cholinergic and serotonergic ionotropic receptors, Neuroscience and Biobehavioral Reviews 23: 817-843 (1999) and Arias, H. R. & Blanton, M. P., Molecular and physicochemical aspects of local anesthetics acting on nicotinic acetylcholine receptor-containing membranes, Mini Reviews in Medicinal Chemistry 2: 385-410 (2002).

For example, tetracaine binds to the receptor channels preferentially in the resting state. Dissociative anesthetics inhibit several neuronal-type nicotinic receptors at clinical concentration ranges, with examples such as phencyclidine (PCP) (Connolly, J., Boulter, J., & Heinemann, S. F., Alpha 4-beta 2 and other nicotinic acetylcholine receptor subtypes as targets of psychoactive and addictive drugs, British Journal of Pharmacology 105: 657-666 (1992)), ketamine (Flood, P. & Krasowski M. D., Intravenous anesthetics differentially modulate ligand-gated ion channels, Anesthesiology 92: 1418-1425 (2000); and Ho, K. K. & Flood, P., Single amino acid residue in the extracellular portion of transmembrane segment 2 in the nicotinic α7 acetylcholine receptor modulates sensitivity to ketamine, Anesthesiology 100: 657-662 (2004)), and dizocilpine (Krasowski, M. D., & Harrison, N. L., General anaesthetic actions on ligand-gated ion channels, Cellular and Molecular Life Sciences 55: 1278-1303 (1999)). Studies indicate that the dissociative anesthetics bind to a single or overlapping sites in the resting ion channel, and suggest that the ketamine/PCP locus partially overlaps the tetracaine binding site in the receptor channel. Dizocilpine, also known as MK-801, is a dissociative anesthetic and anticonvulsant which also acts as a non-competitive antagonist at different nicotinic receptors. Dizocilpine is reported to be an open-channel blocker of α4β2 neuronal nicotinic receptors. See, Buisson, B., & Bertrand, D., Open-channel blockers at the human α4β2 neuronal nicotinic acetylcholine receptor, Molecular Pharmacology 53: 555-563 (1998).

In addition to their well-known actions on monoamine and serotonin reuptake systems, antidepressants have also been shown to modulate nicotinic receptors. Early studies showed that tricyclic antidepressants act as non-competitive antagonists. See, Gumilar, F., Arias, H. R., Spitzmaul, G., Bouzat, C., Molecular mechanisms of inhibition of nicotinic acetylcholine receptors by tricyclic antidepressants. Neuropharmacology 45: 964-76 (2003). Garćia-Colunga et al., report that fluoxetine, a selective serotonin reuptake inhibitor (SSRI), inhibits membrane currents elicited by activation of muscle or neuronal nicotinic receptors in a non-competitive manner; either by increasing the rate of desensitization and/or by inducing channel blockade. See, Garćia-Colunga, J., Awad, J. N., & Miledi, R., Blockage of muscle and neuronal nicotinic acetylcholine receptors by fluoxetine (Prozac), Proceedings of the National Academy of Sciences USA 94: 2041-2044 (1997); and Garćia-Colunga, J., Vazquez-Gomez, E., & Miledi, R., Combined actions of zinc and fluoxetine on nicotinic acetylcholine receptors, The Pharmacogenomics Journal 4: 388-393 (2004). Mecamylamine, a classical non-competitive nicotinic receptor antagonist, is also well known to inhibit receptor function by blocking the ion channel. See, Giniatullin, R. A., Sokolova, E. M., Di Angelantonio, S., Skorinkin, A., Talantova, M. V., Nistri, A. Rapid Relief of Block by Mecamylamine of Neuronal Nicotinic Acetylcholine Receptors of Rat Chromaffin Cells In Vitro: An Electrophysiological and Modeling Study. Molecular Pharmacology 58: 778-787 (2000).

### SUMMARY OF THE INVENTION

The present invention includes compounds of Formulas I, II, and III:

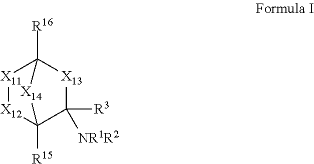

wherein

each of R¹ and R² individually is H, C₁₋₆ alkyl, or R¹ and R² combine with the nitrogen atom to which they are attached to form a 3- to 8-membered ring, which ring may be optionally substituted;

each of R¹⁵ and R¹⁶ individually is H, halogen, C₁₋₆ alkyl, C₁₋₆ haloalkyl, hydroxyl, C₁₋₆ alkoxy, or C₆₋₁₄ aryloxy;

R³ is H or C₁₋₆ alkyl;

each of X¹¹, X¹², X¹³, and X¹⁴ individually is —(CR⁴R⁵)—, where each of R⁴ and R⁵ is individually H, halogen, C₁₋₆ alkyl, C₁₋₆ haloalkyl, hydroxyl, C₁₋₆ alkoxy, or C₆₋₁₄ aryloxy;

or a pharmaceutically acceptable salt thereof.

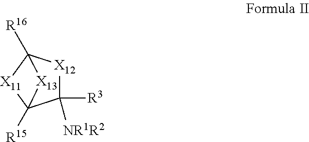

wherein

each of R¹ and R² individually is H, C₁₋₆ alkyl, or R¹ and R² combine with the nitrogen atom to which they are attached to form a 3- to 8-membered ring, which ring may be optionally substituted;

each of R¹⁵ and R¹⁶ individually is H, halogen, C₁₋₆ alkyl, C₁₋₆ haloalkyl, hydroxyl, C₁₋₆ alkoxy, or C₆₋₁₄ aryloxy;

R³ is H or C₁₋₆ alkyl;

each of X¹¹, X¹², and X¹³ individually is —(CR⁴R⁵)—, where each of R⁴ and R⁵ is individually is H, halogen, C₁₋₆ alkyl, C₁₋₆ haloalkyl, hydroxyl, C₁₋₆ alkoxy, or C₆₋₁₄ aryloxy;

or a pharmaceutically acceptable salt thereof.

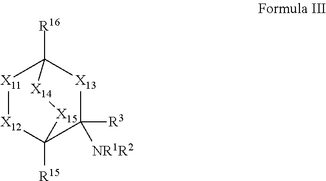

wherein

each of R¹ and R² individually is H, C₁₋₆ alkyl, or R¹ and R² combine with the nitrogen atom to which they are attached to form a 3- to 8-membered ring, which ring may be optionally substituted;

each of R¹⁵ and R¹⁶ individually is H, halogen, C₁₋₆ alkyl, C₁₋₆ haloalkyl, hydroxyl, C₁₋₆ alkoxy, or C₆₋₁₄ aryloxy;

R³ is H or C₁₋₆ alkyl;

each of X¹¹, X¹², X¹³, X¹⁴, and X¹⁵ individually is —(CR⁴R⁵)—, where each of R⁴ and R⁵ is individually is H, halogen, C₁₋₆ alkyl, C₁₋₆ haloalkyl, hydroxyl, C₁₋₆ alkoxy, or C₆₋₁₄ aryloxy;

or a pharmaceutically acceptable salt thereof.

The present invention also includes compounds as represented by Formulae IV, V, VI, and VII:

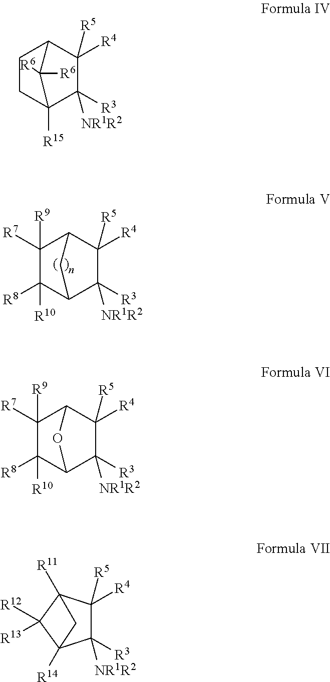

wherein

each of R¹ and R² individually is H, C₁₋₆ alkyl, or R¹ and R² combine with the nitrogen atom to which they are attached to form a 3- to 8-membered ring, which ring may be optionally substituted;

each of R³, R⁶, R¹¹, R¹², R¹³, and R¹⁴ is H or C₁₋₆ alkyl;

n is 1 or 2;

each of R⁴, R⁵, R⁷, R⁸, R⁹, and R¹⁰ individually is H, halogen, C₁₋₆ alkyl, C₁₋₆ haloalkyl, hydroxyl, C₁₋₆ alkoxy, or C₆₋₁₄ aryloxy;

R¹⁵ is H or methyl;

or a pharmaceutically acceptable salt thereof.

Preferably, optionally substituted includes substitution with one or more C₁₋₆ alkyl, halogen, C₁₋₆ haloalkyl, C₁₋₆ alkoxy, or C₆₋₁₄ aryloxy.

The present invention includes pharmaceutical compositions comprising a compound of the present invention or a pharmaceutically acceptable salt thereof. The pharmaceutical compositions of the present invention can be used for treating or preventing a wide variety of conditions or disorders, and particularly those disorders characterized by dysfunction of nicotinic cholinergic neurotransmission or the degeneration of the nicotinic cholinergic neurons.

The present invention includes a method for treating or preventing disorders and dysfunctions, such as CNS disorders and dysfunctions, and also for treating or preventing certain conditions, for example, alleviating pain and inflammation, in mammals in need of such treatment. The methods involve administering to a subject a therapeutically effective amount of a compound of the present invention, including a salt thereof, or a pharmaceutical composition that includes such compounds.

### DETAILED DESCRIPTION OF THE INVENTION

### I. Compounds

The present invention includes compounds of Formulas I, II, and III:

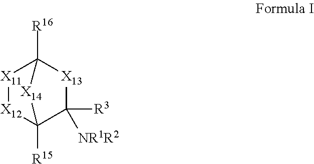

wherein

each of R¹ and R² individually is H, C₁₋₆ alkyl, or R¹ and R² combine with the nitrogen atom to which they are attached to form a 3- to 8-membered ring, which ring may be optionally substituted;

each of R¹⁵ and R¹⁶ individually is H, halogen, C₁₋₆ alkyl, C₁₋₆ haloalkyl, hydroxyl, C₁₋₆ alkoxy, or C₆₋₁₄ aryloxy;

R³ is H or C₁₋₆ alkyl;

each of X¹¹, X¹², X¹³, and X¹⁴ individually is —(CR⁴R⁵)—, where each of R⁴ and R⁵ is individually H, halogen, C₁₋₆ alkyl, C₁₋₆ haloalkyl, hydroxyl, C₁₋₆ alkoxy, or C₆₋₁₄ aryloxy;

or a pharmaceutically acceptable salt thereof.

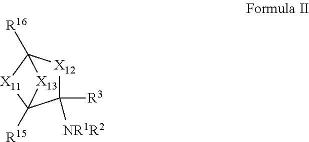

wherein

each of R¹ and R² individually is H, C₁₋₆ alkyl, or R¹ and R² combine with the nitrogen atom to which they are attached to form a 3- to 8-membered ring, which ring may be optionally substituted;

each of R¹⁵ and R¹⁶ individually is H, halogen, C₁₋₆ alkyl, C₁₋₆ haloalkyl, hydroxyl, C₁₋₆ alkoxy, or C₆₋₁₄ aryloxy;

R³ is H or C₁₋₆ alkyl;

each of X¹¹, X¹², and X¹³ individually is —(CR⁴R⁵)—, where each of R⁴ and R⁵ is individually is H, halogen, C₁₋₆ alkyl, C₁₋₆ haloalkyl, hydroxyl, C₁₋₆ alkoxy, or C₆₋₁₄ aryloxy;

or a pharmaceutically acceptable salt thereof.

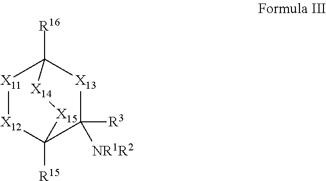

wherein

each of R¹ and R² individually is H, C₁₋₆ alkyl, or R¹ and R² combine with the nitrogen atom to which they are attached to form a 3- to 8-membered ring, which ring may be optionally substituted;

each of R¹⁵ and R¹⁶ individually is H, halogen, C₁₋₆ alkyl, C₁₋₆ haloalkyl, hydroxyl, C₁₋₆ alkoxy, or C₆₋₁₄ aryloxy;

R³ is H or C₁₋₆ alkyl;

each of X¹¹, X¹², X¹³, X¹⁴, and X¹⁵ individually is —(CR⁴R⁵)—, where each of R⁴ and R⁵ is individually is H, halogen, C₁₋₆ alkyl, C₁₋₆ haloalkyl, hydroxyl, C₁₋₆ alkoxy, or C₆₋₁₄ aryloxy;

or a pharmaceutically acceptable salt thereof.

The present invention also includes compounds as represented by Formulae IV, V, VI, and VII:

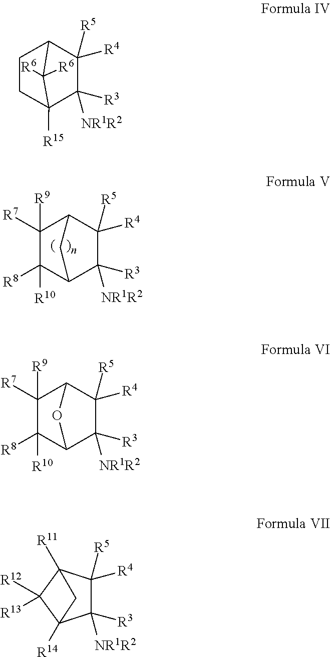

wherein

each of R¹ and R² individually is H, C₁₋₆ alkyl, or R¹ and R² combine with the nitrogen atom to which they are attached to form a 3- to 8-membered ring, which ring may be optionally substituted;

each of R³, R⁶, R¹¹, R¹², R¹³, and R¹⁴ is H or C₁₋₆ alkyl;

n is 1 or 2;

each of R⁴, R⁵, R⁷, R⁸, R⁹, and R¹⁰ individually is H, halogen, C₁₋₆ alkyl, C₁₋₆ haloalkyl, hydroxyl, C₁₋₆ alkoxy, or C₆₋₁₄ aryloxy;

R¹⁵ is H or methyl;

or a pharmaceutically acceptable salt thereof.

Preferably, optionally substituted includes substitution with one or more C₁₋₆ alkyl, halogen, C₁₋₆ haloalkyl, C₁₋₆ alkoxy, or C₆₋₁₄ aryloxy.

One aspect of the present invention includes a pharmaceutical composition comprising a compound of the present invention and a pharmaceutically acceptable carrier.

One aspect of the present invention includes a method for the treatment or prevention of a disease or condition mediated by a neuronal nicotinic receptor, specifically through the use of non-competitive antagonists, including but not limited channel blockers, comprising the administration of a compound of the present invention. In one embodiment, the disease or condition is a CNS disorder. In another embodiment, the disease or condition is inflammation or an inflammatory response associated with one or more of a bacterial or viral infection. In another embodiment, the disease or condition is pain. In another embodiment, the disease or condition is neovascularization. In another embodiment, the disease or condition is hypertension. In another embodiment, the disease or condition is another disorder described herein.

One aspect of the present invention includes use of a compound of the present invention for the preparation of a medicament for the treatment or prevention of a disease or condition mediated by a neuronal nicotinic receptor, specifically through the use of non-competitive antagonists, such as channel blockers. In one embodiment, the disease or condition is a CNS disorder. In another embodiment, the disease or condition is inflammation or an inflammatory response associated with one or more of a bacterial or viral infection. In another embodiment, the disease or condition is pain. In another embodiment, the disease or condition is neovascularization. In another embodiment, the disease or condition is hypertension. In another embodiment, the disease or condition is another disorder described herein.

One aspect of the present invention includes a compound of the present invention for use as an active therapeutic substance. One aspect, thus, includes a compound of the present invention for use in the treatment or prevention of a disease or condition mediated by a neuronal nicotinic receptor, specifically through the use of non-competitive antagonists, such as channel blockers. In one embodiment, the disease or condition is a CNS disorder. In another embodiment, the disease or condition is inflammation or an inflammatory response associated with one or more of a bacterial or viral infection. In another embodiment, the disease or condition is pain. In another embodiment, the disease or condition is neovascularization. In another embodiment, the disease or condition is hypertension. In another embodiment, the disease or condition is another disorder described herein.

The scope of the present invention includes all combinations of aspects and embodiments.

The following definitions are meant to clarify, but not limit, the terms defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “C_{x-y} alkyl,” which refers to an alkyl group, as herein defined, containing the specified number of carbon atoms. Similar terminology will apply for other preferred terms and ranges as well. Thus, for example, C₁₋₆ alkyl represents a straight or branched chain hydrocarbon containing one to six carbon atoms.

As used herein the term “alkyl” refers to a straight or branched chain hydrocarbon, which may be optionally substituted, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used herein, the terms “methylene,” “ethylene,” and “ethenylene,” refer to divalent forms —CH₂—, —CH₂—CH₂—, and —CH═CH—.

As used herein, the term “cycloalkyl” refers to a fully saturated optionally substituted monocyclic, bicyclic, or bridged hydrocarbon ring, with multiple degrees of substitution being allowed. Exemplary “cycloalkyl” groups as used herein include, but are not limited to, cyclopropyl, cyclobutyl, cyclopentyl, cyclohexyl, and cycloheptyl.

As used herein, the term “heterocycle” or “heterocyclyl” refers to an optionally substituted mono- or polycyclic ring system, optionally containing one or more degrees of unsaturation, and also containing one or more heteroatoms, which may be optionally substituted, with multiple degrees of substitution being allowed. Exemplary heteroatoms include nitrogen, oxygen, or sulfur atoms, including N-oxides, sulfur oxides, and dioxides. Preferably, the ring is three to twelve-membered, preferably three- to eight-membered and is either fully saturated or has one or more degrees of unsaturation. Such rings may be optionally fused to one or more of another heterocyclic ring(s) or cycloalkyl ring(s). Examples of “heterocyclic” groups as used herein include, but are not limited to, tetrahydrofuran, pyran, tetrahydropyran, 1,4-dioxane, 1,3-dioxane, piperidine, pyrrolidine, morpholine, tetrahydrothiopyran, and tetrahydrothiophene.

As used herein, the term “aryl” refers to a single benzene ring or fused benzene ring system which may be optionally substituted, with multiple degrees of substitution being allowed. Examples of “aryl” groups as used include, but are not limited to, phenyl, 2-naphthyl, 1-naphthyl, anthracene, and phenanthrene. Preferable aryl rings have five- to ten-members.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with an aromatic ring (aryl, such as a benzene ring) to form, for example, groups such as indanyl and acenaphthalenyl, and also includes such groups as, for non-limiting examples, dihydronaphthalene and tetrahydronaphthalene.

As used herein, the term “heteroaryl” refers to a monocyclic three to seven membered aromatic ring, or to a fused bicyclic aromatic ring system comprising two of such aromatic rings, which may be optionally substituted, with multiple degrees of substitution being allowed. Preferably, such rings contain five- to ten-members. These heteroaryl rings contain one or more nitrogen, sulfur, and/or oxygen atoms, where N-oxides, sulfur oxides, and dioxides are permissible heteroatom substitutions. Examples of “heteroaryl” groups as used herein include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinoxaline, benzofuran, benzoxazole, benzothiophene, indole, indazole, benzimidazole, imidazopyridine, pyrazolopyridine, and pyrazolopyrimidine.

As used herein the term “halogen” refers to fluorine, chlorine, bromine, or iodine.

As used herein the term “haloalkyl” refers to an alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein the term “alkoxy” refers to a group —OR^{a}, where R^{a} is alkyl as herein defined. Likewise, the term “alkylthio” refers to a group —SR^{a}, where R^{a} is alkyl as herein defined.

As used herein the term “aryloxy” refers to a group —OR^{a}, where R^{a} is aryl as herein defined. Likewise, the term “arylthio” refers to a group —SR^{a}, where R^{a} is aryl as herein defined.

As used herein “amino” refers to a group —NR^{a}R^{b}, where each of R^{a} and R^{b} is hydrogen. Additionally, “substituted amino” refers to a group —NR^{a}R^{b} wherein each of R^{a} and R^{b} individually is alkyl, alkenyl, alkynyl, cycloalkyl, aryl, heterocylcyl, or heteroaryl. As used herein, when either R^{a} or R^{b} is other than hydrogen, such a group may be referred to as a “substituted amino” or, for example if R^{a} is H and R^{b} is alkyl, as an “alkylamino.”

As used herein, the term “pharmaceutically acceptable” refers to carrier(s), diluent(s), excipient(s) or salt forms of the compounds of the present invention that are compatible with the other ingredients of the formulation and not deleterious to the recipient of the pharmaceutical composition.

As used herein, the term “pharmaceutical composition” refers to a compound of the present invention optionally admixed with one or more pharmaceutically acceptable carriers, diluents, or excipients. Pharmaceutical compositions preferably exhibit a degree of stability to environmental conditions so as to make them suitable for manufacturing and commercialization purposes.

As used herein, the terms “effective amount”, “therapeutic amount”, and “effective dose” refer to an amount of the compound of the present invention sufficient to elicit the desired pharmacological or therapeutic effects, thus resulting in an effective treatment of a disorder. Treatment of a disorder may be manifested by delaying or preventing the onset or progression of the disorder, as well as the onset or progression of symptoms associated with the disorder. Treatment of a disorder may also be manifested by a decrease or elimination of symptoms, reversal of the progression of the disorder, as well as any other contribution to the well being of the patient.

The effective dose can vary, depending upon factors such as the condition of the patient, the severity of the symptoms of the disorder, and the manner in which the pharmaceutical composition is administered. Typically, to be administered in an effective dose, compounds may be administered in an amount of less than 5 mg/kg of patient weight. The compounds may be administered in an amount from less than about 1 mg/kg patient weight to less than about 100 μg/kg of patient weight, and further between about 1 μg/kg to less than 100 μg/kg of patient weight. The foregoing effective doses typically represent that amount that may be administered as a single dose, or as one or more doses that may be administered over a 24 hours period.

The compounds of this invention may be made by a variety of methods, including well-established synthetic methods. Illustrative general synthetic methods are set out below and then specific compounds of the invention are prepared in the working Examples.

In the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3^{rd} Edition, John Wiley & Sons, herein incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

The present invention also provides a method for the synthesis of compounds useful as intermediates in the preparation of compounds of the present invention along with methods for their preparation.

The compounds can be prepared according to the methods described below using readily available starting materials and reagents. In these reactions, variants may be employed which are themselves known to those of ordinary skill in this art but are not described in detail here.

Unless otherwise stated, structures depicted herein are also meant to include compounds which differ only in the presence of one or more isotopically enriched atoms. Compounds having the present structure except for the replacement of one or more hydrogen atoms by deuterium or tritium atoms, or the replacement of one or more carbon atoms by a ¹³C- or ¹⁴C-enriched carbon atoms are within the scope of the invention. For example, deuterium has been widely used to examine the pharmacokinetics and metabolism of biologically active compounds. Although deuterium behaves similarly to hydrogen from a chemical perspective, there are significant differences in bond energies and bond lengths between a deuterium-carbon bond and a hydrogen-carbon bond. Consequently, replacement of hydrogen by deuterium in a biologically active compound may result in a compound that generally retains its biochemical potency and selectivity but manifests significantly different absorption, distribution, metabolism, and/or excretion (ADME) properties compared to its isotope-free counterpart. Thus, deuterium substitution may result in improved drug efficacy, safety, and/or tolerability for some biologically active compounds.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted.

When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds (Wiley-Interscience, 1994).

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

### II. General Synthetic Methods

Those skilled in the art of organic synthesis will appreciate that there exist multiple means of producing compounds of the present invention, as well as means for producing compounds of the present invention which are labeled with a radioisotope appropriate to various uses.

Compounds of Formula IV provide a general structural scaffold that is representative of the compounds of the present invention. Compounds of Formula IV can be made in a variety of ways. For instance, as shown in Scheme 1, each of fenchone (either enantiomer) and camphor (either enantiomer), all of which are commercially available, provide a ready entry to compounds of Formula IV. Likewise, norbornanone and its derivatives, known in the chemical literature, could also be used as starting materials. As shown in Scheme 1, the reaction sequence involves optional alkylation (via the corresponding enolate) adjacent to the ketone carbonyl, followed by Wittig transformation of the ketone into the corresponding methylene alkene. Conversion of the methylene alkene into the amine can be accomplished using a Ritter-type reaction, followed by reduction with a metal hydride reducing agent (see, for instance, U.S. Pat. No. 5,986,142). A variety of other reagents, known to those of skill in the art of organic synthesis, can be used to accomplish each of the steps in Scheme 1. For instance, a variety of the alkyl halides (R⁴X and R⁵X in Scheme 1) can be used for the alkylation reactions. The alkylation reaction can be run either once or twice, resulting in either mono- or di-alkylation adjacent to the carbonyl. Also, the alkylation of the secondary amine to give the tertiary amine can be accomplished using a variety of alkyl halides (R²X in Scheme 1). In a variation not shown in Scheme 1, an organometallic reagent (e.g., an alkyllithium or a Grignard reagent) can be reacted with the ketone to give the expected tertiary alcohol, which can then be transformed by Ritter reaction conditions to the compounds of Formula IV (in which R³ varies according to the nature of the organometallic reagent used).

While the derivatives shown in Scheme 1 derive largely from alkylation reactions at either nitrogen or carbon, other transformations of the ketone intermediate are possible. For instance, substitution of one or more of the hydrogen atoms adjacent to the carbonyl functionality (i.e., alpha substitution) with fluorine atoms can be accomplished using a variety of reagent combinations, usually through the intermediacy of an enolate. Thus, reaction of norbornanone or camphor with lithium diisopropylamide (LDA) or sodium hexamethyldisilazide (to form the enolate), followed by reaction of the enolate with N-fluorobenzenesulfonimide, will produce the corresponding alpha-fluoro ketones (3-fluorobicyclo[2.2.1]heptan-2-one and 1,7,7-trimethyl-3-fluorobicyclo[2.2.1]heptan-2-one, respectively) (see, for instance, Suzuki et al., J. Org. Chem. 72(1): 246 (2007)). These alpha-fluoro ketones can be further transformed according to the reactions illustrated in Scheme 1.

Enolizable ketones, such as norbornanone or camphor, can also be converted into alpha-alkoxy ketones, using a variety or reagents and conditions. Commonly, the ketone is first converted into an enol ether, and then treated with an oxidizing agent in the presence of an alcohol. For instance, treatment of norbornanone or camphor with trimethyl orthoformate and catalytic p-toluenesulfonic acid in methanol can be used to make the corresponding methyl enol ethers, and treatment of these intermediates with fluorine gas in methanol will provide (3-methoxybicyclo[2.2.1]heptan-2-one and 1,7,7-trimethyl-3-methoxybicyclo[2.2.1]heptan-2-one, respectively (see, for instance, Rozen et al., J. Amer. Chem. Soc. 114(20): 7643 (1992)). Other similar reaction sequences, involving the intermediacy of ether enol ethers and/or epoxides, are also known to those of skill in the art to be useful in making alpha-alkoxy ketones. These alpha-alkoxy ketones can be further transformed according to the reactions illustrated in Scheme 1.

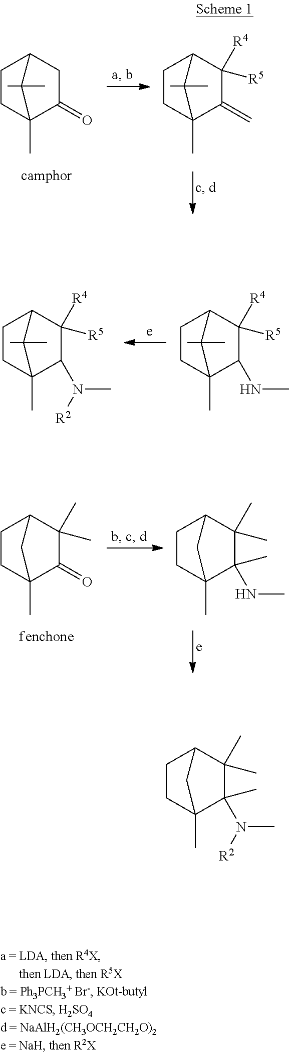

Compounds of Formulae IV and V can be made from Diels-Alder adducts, as shown in Schemes 2 and 3 respectively. Thus, as shown in Scheme 2,5-nitrobicyclo[2.2.1]hept-2-ene (the adduct of cyclopentadiene and nitroethylene) can be hydrogenated, to give the nitroalkane, and then optionally alkylated adjacent to the nitro group, using alkoxide base and alkyl halides (R³X in Scheme 2). The nitro compounds thus produced can then be reduced to the corresponding primary amines using standard conditions (typically tin metal in HCl, or iron filings in acetic acid), and the primary amines can then be converted to the corresponding secondary and tertiary amines using standard methodologies, generally employing a base and an alkyl halide (R¹X and R²X in Scheme 2) in each alkylation step.

Varying the reactants in the Diels-Alder reaction results in other adducts which are also useful as starting materials for synthesis of compounds of Formulas IV and V. Thus, the use of 5,5-dialkylcyclopentadienes gives rise to the corresponding 7,7-dialkyl-5-nitrobicyclo[2.2.1]hept-2-enes (Scheme 2, R⁶ is alkyl) (see, for instance, Eilbracht et al., Tetrahedron Lett. 2225-2228 (1976) and Burnell et al., Can. J. Chem. 65(1): 154 (1987). The reactions described in the preceding paragraph (hydrogenation of the alkene, alkylation adjacent to the nitro group, reduction of the nitro group to the amine, and alkylation of the primary amine to give secondary and tertiary amines) can be performed on these 7,7-dialkyl Diels-Alder adducts, producing compounds of Formula IV.

The dieneophile can also be varied. Thus, 1-nitropropene and 1-nitro-2-methylpropene also react with cyclopentadiene to give the corresponding methylated Diels-Alder adducts, 6-methyl-5-nitrobicyclo[2.2.1]hept-2-ene and 6,6-dimethyl-5-nitrobicyclo[2.2.1]hept-2-ene (see, for instance, Noyce, J. Amer. Chem. Soc. 73: 20 (1951) and Van Tamelen and Thiede, J. Amer. Chem. Soc. 74: 2615 (1952). These nitroalkenes can be utilized similarly to 5-nitrobicyclo[2.2.1]hept-2-ene in the generation of compounds of Formula IV.

As shown in Scheme 3, Diels-Alder adducts of the nitroethylene+cyclic diene kind can be used to access a variety of compounds of Formula IV. For instance, reaction of 1-nitro-2-methylpropene with 1,3-cyclohexadiene will generate 6,6-dimethyl-5-nitrobicyclo[2.2.2]oct-2-ene, a starting material for the reactions shown in Scheme 3. Thus, 6,6-dimethyl-5-nitrobicyclo[2.2.2]oct-2-ene can be hydrogenated to give 3,3-dimethyl-2-nitrobicyclo[2.2.2]octane and subsequently converted, via the Nef reaction, to 3,3-dimethylbicyclo[2.2.2]octan-2-one. As illustrated in Schemes 1 and 2, each of the latter two compounds can be further transformed to give intermediates useful in the synthesis of compounds of Formula IV. For instance, 3,3-dimethyl-2-nitrobicyclo[2.2.2]octane can be alkylated adjacent to the nitro group (chemistry that is illustrated in Scheme 2), and dimethylbicyclo[2.2.2]octan-2-one can be converted into the corresponding exocyclic alkene (chemistry that is illustrated in Scheme 1). Each of these products can then be further transformed, using chemistry illustrated in Schemes 1 and 2, to compounds of Formula IV.

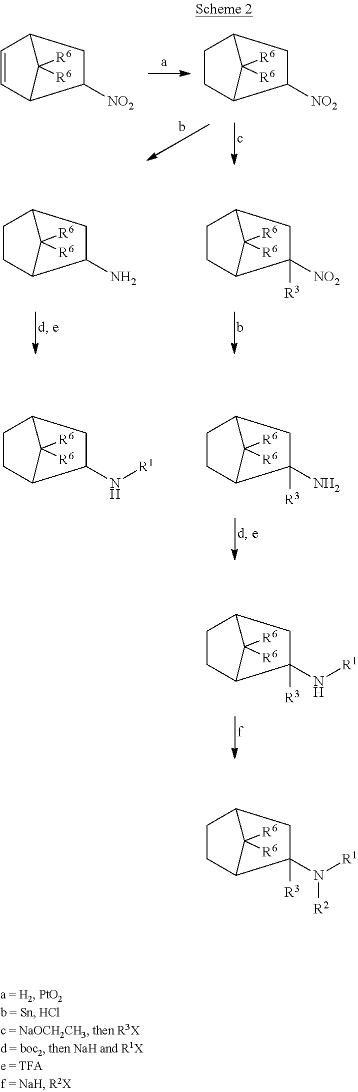

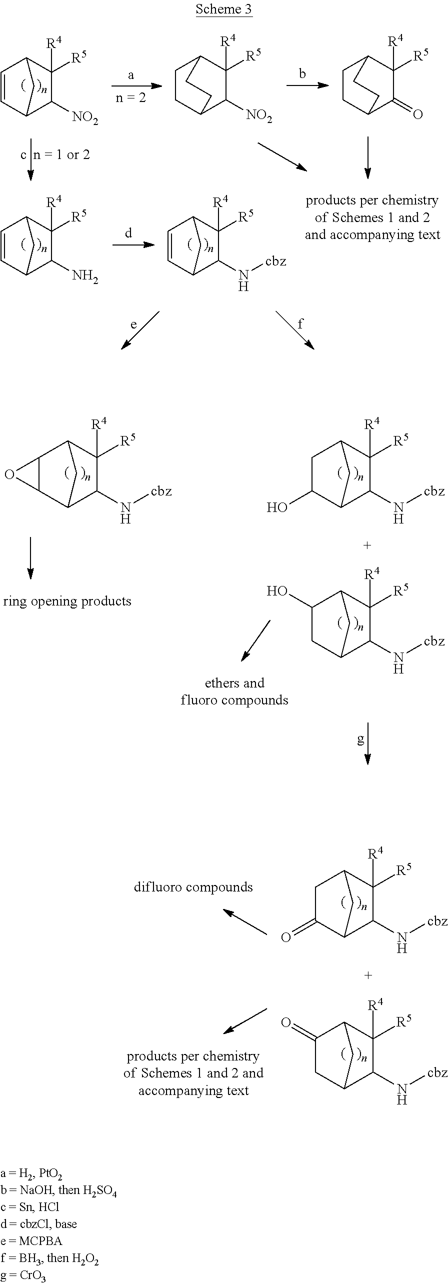

A variety of substituent groups can be installed, at various positions, in either the bicyclo[2.2.1]heptane or bicyclo[2.2.2]octane examples of compounds of Formula IV. For example, as shown in Scheme 3, the Diels-Alder adducts (nitro compounds) of either ring size (n=1 and 2, respectively; R⁴ and R⁵ defined as before) can be reduced to the corresponding amine compounds (by treatment with tin and hydrochloric acid) which are then protected as their benzylcarbamates (by treatment with benzylchloroformate and base). The alkene functionality can then be used to install various substituents on the ring, through reactions characteristic of alkenes. For example, as shown in Scheme 3, reaction of the alkene containing, benzylcarbamate (cbz) protected amine with m-chloroperoxybenzoic acid (or some similar peroxyacid) will produce the corresponding epoxide, which can then react with various nucleophiles to produce compounds resulting from epoxide ring opening. Such nucleophiles include fluoride, alkoxide and aryloxide, producing fluoro alcohols, alkoxy alcohols and aryloxy alcohols, respectively. De-protection of the amine functionality (removal of the cbz protecting group) then leads to compounds of Formula V.

In another example of the utility of the alkene moiety in the installation of substituents, reaction of the alkene containing, benzylcarbamate protected amine with borane, followed by hydrogen peroxide, will produce regioisomeric alcohols (as shown in Scheme 3). These can subsequently be oxidized to give the corresponding ketones. The alcohols can be converted into either fluoro compounds or ethers of various kinds. Removal of the cbz group (typically accomplished by hydrogenation) will produce compounds of Formula V. The ketones can be converted, using chemistry illustrated in Schemes 1 and 2, into a variety of intermediates which, upon de-protection of the amino group, become compounds of Formula V. The ketone intermediates can also be reacted with sulfur tetrafluoride or (diethylamino)sulfur trifluoride (DAST) (for example, see Golubev et al., Tetrahedron Lett. 45: 1445 (2004)), producing the corresponding geminal difluorides.

Yet another variation on the Diels-Alder reaction makes use of furan as the diene component. Thus, as described by Eggelte at al. (Heterocycles 4(1): 19-22 (1976)), reaction of nitroethylene with furan gives 5-nitro-7-oxabicyclo[2.2.1]hept-2-ene, which can then be converted into the saturated nitroalkane (2-nitro-7-oxabicyclo[2.2.1]heptane) and the ketone (7-oxabicyclo[2.2.1]heptan-2-one) (see Scheme 4). As illustrated in Schemes 1 and 2 (and described in the accompanying text), the nitroalkane and ketone intermediates can be transformed into a variety of compounds using reactions well known to those of skill in organic synthesis. In the case of the nitroalkane and ketone intermediates derived from furan (containing the bridging oxygen), chemistry similar to that shown in Schemes 1 and 2 will produce compounds of Formula VI (see Scheme 4). Also, the alkene functionality can be used introduce substituents, as illustrated in Scheme 3 and described in the accompanying text. Thus, chemistry similar to that shown in Scheme 3 can be performed on the oxygen bridged intermediates to generate compounds of Formula VI (see Scheme 4). Finally, it is also worth noting that commercially available materials, such as cantharidic acid, can serve as starting materials for synthesis of compounds of Formula VI.

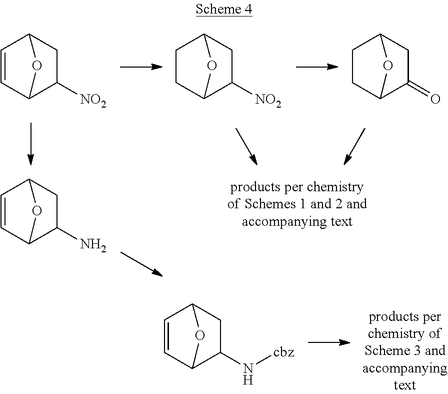

Compounds of Formula VII can be made as illustrated in Scheme 5. Thus, as reported by Wolff and Agosta, J. Amer. Chem. Soc. 105(5): 1292 (1983), Crimmins and Reinhold, Organic Reactions 44 (1993), and others, ultraviolet irradiation (typically >340 nm) of 1,5-hexadien-3-ones produces bicyclo[2.1.1]hexan-2-ones. This photochemical 2+2 cycloaddition is quite tolerant to alkyl substituents on the alkene moieties of the 1,5-hexadien-3-ones (R¹¹, R¹², R¹³ and R¹⁴ in Scheme 5), and the bicyclo[2.1.1]hexan-2-ones thus produced can be transformed, using chemistry illustrated in Scheme 1 and described in the accompanying text (e.g., alkylations alpha to the ketone carbonyl, Wittig olefination, addition of an organometallic reagent to the carbonyl, Ritter reaction, alkylation of primary to secondary and tertiary amines), into compounds of Formula VII.

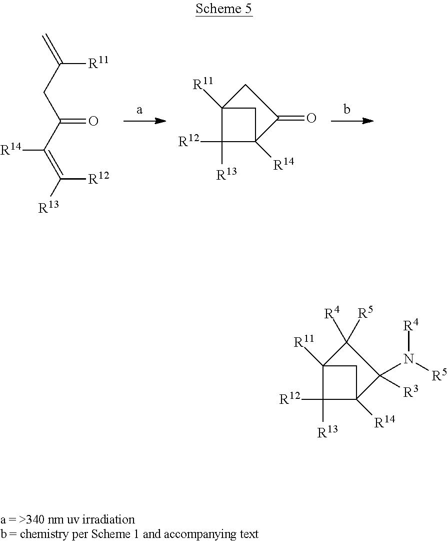

The chemistry in Schemes 1-5 and the accompanying text is illustrative of that which can be used to produce compounds of Formulas I-VII. Such chemistry can be employed in a variety of reaction sequences, including but not restricted by, those expressly drawn or described. Other analogous chemistry, also well known to those of skill in the art of organic synthesis, can be used to make compounds of Formulas I-VII. It is also noteworthy that reagents incorporating various isotopes, both stable and radioactive, of the atoms involved can be used. Thus, in the case of compounds of Formulas I-VII, those analogs incorporating such isotopes as deuterium (D or ²H), tritium (T or ³H), ¹³C, ¹⁴C, ¹⁵N, ¹⁸O, and ¹⁸F can be accomplished. Reagents containing one or more deuterium atoms are particularly readily available, either commercially (e.g., iodomethane, methyllithium, deuterium gas, various metal deuteride reducing agents) or via routine transformation of commercially available materials. Thus, the incorporation of deuterium into compounds of Formulas I-VII is particularly straightforward.

### III. Pharmaceutical Compositions

Although it is possible to administer the compound of the present invention in the form of a bulk active chemical, it is preferred to administer the compound in the form of a pharmaceutical composition or formulation. Thus, one aspect the present invention includes pharmaceutical compositions comprising one or more compounds of Formulas I-VII and/or pharmaceutically acceptable salts thereof and one or more pharmaceutically acceptable carriers, diluents, or excipients. Another aspect of the invention provides a process for the preparation of a pharmaceutical composition including admixing one or more compounds of Formulas I-VII and/or pharmaceutically acceptable salts thereof with one or more pharmaceutically acceptable carriers, diluents or excipients.

The manner in which the compound of the present invention is administered can vary. The compound of the present invention is preferably administered orally. Preferred pharmaceutical compositions for oral administration include tablets, capsules, caplets, syrups, solutions, and suspensions. The pharmaceutical compositions of the present invention may be provided in modified release dosage forms such as time-release tablet and capsule formulations.

The pharmaceutical compositions can also be administered via injection, namely, intravenously, intramuscularly, subcutaneously, intraperitoneally, intraarterially, intrathecally, and intracerebroventricularly. Intravenous administration is a preferred method of injection. Suitable carriers for injection are well known to those of skill in the art and include 5% dextrose solutions, saline, and phosphate buffered saline.

The formulations may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation), by powder injection, or by buccal, sublingual, or intranasal absorption.

Pharmaceutical compositions may be formulated in unit dose form, or in multiple or subunit doses

The administration of the pharmaceutical compositions described herein can be intermittent, or at a gradual, continuous, constant or controlled rate. The pharmaceutical compositions may be administered to a warm-blooded animal, for example, a mammal such as a mouse, rat, cat, rabbit, dog, pig, cow, or monkey; but advantageously is administered to a human being. In addition, the time of day and the number of times per day that the pharmaceutical composition is administered can vary.

The compounds of the present invention may be used in the treatment of a variety of disorders and conditions and, as such, may be used in combination with a variety of other suitable therapeutic agents useful in the treatment or prophylaxis of those disorders or conditions. Thus, one embodiment of the present invention includes the administration of the compound of the present invention in combination with other therapeutic compounds. For example, the compound of the present invention can be used in combination with other NNR ligands (such as varenicline), allosteric modulators of NNRs, antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, antidepressants (such as imipramine, fluoxetine, paroxetine, escitalopram, sertraline, venlafaxine, and duloxetine), anxiolytics (such as alprazolam and buspirone), anticonvulsants (such as phenyloin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole). Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present invention includes combination therapy comprising administering to the subject a therapeutically or prophylactically effective amount of the compound of the present invention and one or more other therapy including chemotherapy, radiation therapy, gene therapy, or immunotherapy.

### IV. Method of Using Pharmaceutical Compositions

The compounds of the present invention can be used for the prevention or treatment of various conditions or disorders for which other types of nicotinic compounds have been proposed or are shown to be useful as therapeutics, such as CNS disorders, hypertension, inflammation, inflammatory response associated with bacterial and/or viral infection, pain, metabolic syndrome, autoimmune disorders, addictions, obesity or other disorders described in further detail herein. This compound can also be used as a diagnostic agent (in vitro and in vivo). Such therapeutic and other teachings are described, for example, in references previously listed herein, including Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Bencherif et al., J. Pharmacol. Exp. Ther. 279: 1413 (1996), Lippiello et al., J. Pharmacol. Exp. Ther. 279: 1422 (1996), Damaj et al., J. Pharmacol. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. No. 5,583,140 to Bencherif et al., U.S. Pat. No. 5,597,919 to Dull et al., U.S. Pat. No. 5,604,231 to Smith et al. and U.S. Pat. No. 5,852,041 to Cosford et al.

### CNS Disorders

The compounds and their pharmaceutical compositions are useful in the treatment or prevention of a variety of CNS disorders, including neurodegenerative disorders, neuropsychiatric disorders, neurologic disorders, and addictions. The compounds and their pharmaceutical compositions can be used to treat or prevent cognitive deficits and dysfunctions, age-related and otherwise; attentional disorders and dementias, including those due to infectious agents or metabolic disturbances; to provide neuroprotection; to treat convulsions and multiple cerebral infarcts; to treat mood disorders, compulsions and addictive behaviors; to provide analgesia; to control inflammation, such as mediated by cytokines and nuclear factor kappa B; to treat inflammatory disorders; to provide pain relief; and to treat infections, as anti-infectious agents for treating bacterial, fungal, and viral infections. Among the disorders, diseases and conditions that the compounds and pharmaceutical compositions of the present invention can be used to treat or prevent are: age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, tardive dyskinesia, spastic dystonia, hyperkinesia, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, compulsive overeating and sexual dysfunction.

Cognitive impairments or dysfunctions may be associated with psychiatric disorders or conditions, such as schizophrenia and other psychotic disorders, including but not limited to psychotic disorder, schizophreniform disorder, schizoaffective disorder, delusional disorder, brief psychotic disorder, shared psychotic disorder, and psychotic disorders due to a general medical conditions, dementias and other cognitive disorders, including but not limited to mild cognitive impairment, pre-senile dementia, Alzheimer's disease, senile dementia, dementia of the Alzheimer's type, age-related memory impairment, Lewy body dementia, vascular dementia, AIDS dementia complex, dyslexia, Parkinsonism including Parkinson's disease, cognitive impairment and dementia of Parkinson's Disease, cognitive impairment of multiple sclerosis, cognitive impairment caused by traumatic brain injury, dementias due to other general medical conditions, anxiety disorders, including but not limited to panic disorder without agoraphobia, panic disorder with agoraphobia, agoraphobia without history of panic disorder, specific phobia, social phobia, obsessive-compulsive disorder, post-traumatic stress disorder, acute stress disorder, generalized anxiety disorder and generalized anxiety disorder due to a general medical condition, mood disorders, including but not limited to major depressive disorder, dysthymic disorder, bipolar depression, bipolar mania, bipolar I disorder, depression associated with manic, depressive or mixed episodes, bipolar II disorder, cyclothymic disorder, and mood disorders due to general medical conditions, sleep disorders, including but not limited to dyssomnia disorders, primary insomnia, primary hypersomnia, narcolepsy, parasomnia disorders, nightmare disorder, sleep terror disorder and sleepwalking disorder, mental retardation, learning disorders, motor skills disorders, communication disorders, pervasive developmental disorders, attention-deficit and disruptive behavior disorders, attention deficit disorder, attention deficit hyperactivity disorder, feeding and eating disorders of infancy, childhood, or adults, tic disorders, elimination disorders, substance-related disorders, including but not limited to substance dependence, substance abuse, substance intoxication, substance withdrawal, alcohol-related disorders, amphetamine or amphetamine-like-related disorders, caffeine-related disorders, cannabis-related disorders, cocaine-related disorders, hallucinogen-related disorders, inhalant-related disorders, nicotine-related disorders, opioid-related disorders, phencyclidine or phencyclidine-like-related disorders, and sedative-, hypnotic- or anxiolytic-related disorders, personality disorders, including but not limited to obsessive-compulsive personality disorder and impulse-control disorders. Cognitive performance may be assessed with a validated cognitive scale, such as, for example, the cognitive subscale of the Alzheimer's Disease Assessment Scale (ADAS-cog). One measure of the effectiveness of the compounds of the present invention in improving cognition may include measuring a patient's degree of change according to such a scale.

Regarding compulsions and addictive behaviors, the compounds of the present invention may be used as a therapy for nicotine addiction and for other brain-reward disorders, such as substance abuse including alcohol addiction, illicit and prescription drug addiction, eating disorders, including obesity, and behavioral addictions, such as gambling, or other similar behavioral manifestations of addiction.

The above conditions and disorders are discussed in further detail, for example, in the American Psychiatric Association: Diagnostic and Statistical Manual of Mental Disorders, Fourth Edition, Text Revision, Washington, D.C., American Psychiatric Association, 2000. This Manual may also be referred to for greater detail on the symptoms and diagnostic features associated with substance use, abuse, and dependence.

Preferably, the treatment or prevention of diseases, disorders and conditions occurs without appreciable adverse side effects, including, for example, significant increases in blood pressure and heart rate, significant negative effects upon the gastro-intestinal tract, and significant effects upon skeletal muscle.

Compounds of Formulas I-VII, when employed in effective amounts, are believed to modulate the activity nicotinic receptors by blockade, to various degrees, of the nicotinic ion channel. Thus, the present invention is believed to provide compounds useful as non-competitive channel blockers for a variety of diseases and conditions. These compounds are believed to be relatively selective in their blockade of nicotinic ion channels, such that side effects associated with blockade of other ion channels are avoided. Thus, the present invention provides the use of a compound of the present invention, or a pharmaceutically acceptable salt thereof, for use in therapy, such as a therapy herein described.

In yet another aspect the present invention provides the use of a compound of the present invention, or a pharmaceutically acceptable salt thereof, in the manufacture of a medicament for use in the treatment of a CNS disorder, such as a disorder, disease or condition described hereinabove.

### Inflammation

The nervous system, primarily through the vagus nerve, is known to regulate the magnitude of the innate immune response by inhibiting the release of macrophage tumor necrosis factor (TNF). This physiological mechanism is known as the “cholinergic anti-inflammatory pathway” (see, for example, Tracey, “The Inflammatory Reflex,” Nature 420: 853-9 (2002)). Excessive inflammation and tumor necrosis factor synthesis cause morbidity and even mortality in a variety of diseases. These diseases include, but are not limited to, endotoxemia, rheumatoid arthritis, osteoarthritis, psoriasis, asthma, atherosclerosis, idiopathic pulmonary fibrosis, and inflammatory bowel disease.

Inflammatory conditions that can be treated or prevented by administering the compounds described herein include, but are not limited to, chronic and acute inflammation, psoriasis, endotoxemia, gout, acute pseudogout, acute gouty arthritis, arthritis, rheumatoid arthritis, osteoarthritis, allograft rejection, chronic transplant rejection, asthma, atherosclerosis, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, irritable bowel syndrome, Crohn's disease, ulcers, ulcerative colitis, acute cholangitis, aphthous stomatitis, cachexia, pouchitis, glomerulonephritis, lupus nephritis, thrombosis, and graft vs. host reaction.

Inflammatory Response Associated with Bacterial and/or Viral Infection

Many bacterial and/or viral infections are associated with side effects brought on by the formation of toxins, and the body's natural response to the bacteria or virus and/or the toxins. As discussed above, the body's response to infection often involves generating a significant amount of TNF and/or other cytokines. The over-expression of these cytokines can result in significant injury, such as septic shock (when the bacteria is sepsis), endotoxic shock, urosepsis, viral pneumonitis and toxic shock syndrome.

Cytokine expression is mediated by NNRs, and can be inhibited by administering agonists or partial agonists of these receptors. Those compounds described herein that are agonists or partial agonists of these receptors can therefore be used to minimize the inflammatory response associated with bacterial infection, as well as viral and fungal infections. Examples of such bacterial infections include anthrax, botulism, and sepsis. Some of these compounds may also have antimicrobial properties. Furthermore, the compounds can be used in the treatment of Raynaud's disease, namely viral-induced painful peripheral vasoconstriction.

These compounds can also be used as adjunct therapy in combination with existing therapies to manage bacterial, viral and fungal infections, such as antibiotics, antivirals and antifungals. Antitoxins can also be used to bind to toxins produced by the infectious agents and allow the bound toxins to pass through the body without generating an inflammatory response. Examples of antitoxins are disclosed, for example, in U.S. Pat. No. 6,310,043 to Bundle et al. Other agents effective against bacterial and other toxins can be effective and their therapeutic effect can be complemented by co-administration with the compounds described herein.

### Pain

The compounds can be administered to treat and/or prevent pain, including acute, neurologic, inflammatory, neuropathic and chronic pain. The compounds can be used in conjunction with opiates to minimize the likelihood of opiate addiction (e.g., morphine sparing therapy). The analgesic activity of compounds described herein can be demonstrated in models of persistent inflammatory pain and of neuropathic pain, performed as described in U.S. Published Patent Application No. 20010056084 A1 (Allgeier et al.) (e.g., mechanical hyperalgesia in the complete Freund's adjuvant rat model of inflammatory pain and mechanical hyperalgesia in the mouse partial sciatic nerve ligation model of neuropathic pain).

The analgesic effect is suitable for treating pain of various genesis or etiology, in particular in treating inflammatory pain and associated hyperalgesia, neuropathic pain and associated hyperalgesia, chronic pain (e.g., severe chronic pain, post-operative pain and pain associated with various conditions including cancer, angina, renal or biliary colic, menstruation, migraine, and gout). Inflammatory pain may be of diverse genesis, including arthritis and rheumatoid disease, teno-synovitis and vasculitis. Neuropathic pain includes trigeminal or herpetic neuralgia, neuropathies such as diabetic neuropathy pain, causalgia, low back pain and deafferentation syndromes such as brachial plexus avulsion.

### Neovascularization

The α7 NNR is associated with neovascularization. Inhibition of neovascularization, for example, by administering antagonists (or at certain dosages, partial agonists) of the α7 NNR can treat or prevent conditions characterized by undesirable neovascularization or angiogenesis. Such conditions can include those characterized by inflammatory angiogenesis and/or ischemia-induced angiogenesis. Neovascularization associated with tumor growth can also be inhibited by administering those compounds described herein that function as antagonists or partial agonists of α7 NNR.

Specific antagonism of α7 NNR-specific activity reduces the angiogenic response to inflammation, ischemia, and neoplasia. Guidance regarding appropriate animal model systems for evaluating the compounds described herein can be found, for example, in Heeschen, C. et al., “A novel angiogenic pathway mediated by non-neuronal nicotinic acetylcholine receptors,” J. Clin. Invest. 110(4):527-36 (2002).

Representative tumor types that can be treated using the compounds described herein include SCLC, NSCLC, ovarian cancer, pancreatic cancer, breast carcinoma, colon carcinoma, rectum carcinoma, lung carcinoma, oropharynx carcinoma, hypopharynx carcinoma, esophagus carcinoma, stomach carcinoma, pancreas carcinoma, liver carcinoma, gallbladder carcinoma, bile duct carcinoma, small intestine carcinoma, urinary tract carcinoma, kidney carcinoma, bladder carcinoma, urothelium carcinoma, female genital tract carcinoma, cervix carcinoma, uterus carcinoma, ovarian carcinoma, choriocarcinoma, gestational trophoblastic disease, male genital tract carcinoma, prostate carcinoma, seminal vesicles carcinoma, testes carcinoma, germ cell tumors, endocrine gland carcinoma, thyroid carcinoma, adrenal carcinoma, pituitary gland carcinoma, skin carcinoma, hemangiomas, melanomas, sarcomas, bone and soft tissue sarcoma, Kaposi's sarcoma, tumors of the brain, tumors of the nerves, tumors of the eyes, tumors of the meninges, astrocytomas, gliomas, glioblastomas, retinoblastomas, neuromas, neuroblastomas, Schwannomas, meningiomas, solid tumors arising from hematopoietic malignancies (such as leukemias, chloromas, plasmacytomas and the plaques and tumors of mycosis fungoides and cutaneous T-cell lymphoma/leukemia), and solid tumors arising from lymphomas.

The compounds can also be administered in conjunction with other forms of anti-cancer treatment, including co-administration with antineoplastic antitumor agents such as cis-platin, adriamycin, daunomycin, and the like, and/or anti-VEGF (vascular endothelial growth factor) agents, as such are known in the art.

The compounds can be administered in such a manner that they are targeted to the tumor site. For example, the compounds can be administered in microspheres, microparticles or liposomes conjugated to various antibodies that direct the microparticles to the tumor. Additionally, the compounds can be present in microspheres, microparticles or liposomes that are appropriately sized to pass through the arteries and veins, but lodge in capillary beds surrounding tumors and administer the compounds locally to the tumor. Such drug delivery devices are known in the art.

### Other Disorders

In addition to treating CNS disorders, inflammation, and neovascularization, and pain, the compounds of the present invention can be also used to prevent or treat certain other conditions, diseases, and disorders in which NNRs play a role. Examples include autoimmune disorders such as lupus, disorders associated with cytokine release, cachexia secondary to infection (e.g., as occurs in AIDS, AIDS related complex and neoplasia), obesity, pemphitis, urinary incontinence, overactive bladder, diarrhea, constipation, retinal diseases, infectious diseases, myasthenia, Eaton-Lambert syndrome, hypertension, preeclampsia, osteoporosis, vasoconstriction, vasodilatation, cardiac arrhythmias, type I diabetes, type II diabetes, bulimia, anorexia and sexual dysfunction, as well as those indications set forth in published PCT application WO 98/25619. The compounds of this invention can also be administered to treat convulsions such as those that are symptomatic of epilepsy, and to treat conditions such as syphillis and Creutzfeld-Jakob disease.

Compounds of the present invention may be used to treat bacterial infections and dermatologic conditions, such as pemphigus folliaceus, pemphigus vulgaris, and other disorders, such as acantholysis, where autoimmune responses with high ganglionic NNR antibody titer is present. In these disorders, and in other autoimmune diseases, such as Mysthenia Gravis, the fab fragment of the antibody binds to the NNR receptor (crosslinking 2 receptors), which induces internalization and degradation.

### Diagnostic Uses

The compounds can be used in diagnostic compositions, such as probes, particularly when they are modified to include appropriate labels. For this purpose the compounds of the present invention most preferably are labeled with a radioactive isotopic moiety such as ¹¹C, ¹⁵F, ⁷⁶Br, ¹²³I or ¹²⁵I.

The administered compounds can be detected using known detection methods appropriate for the label used. Examples of detection methods include position emission topography (PET) and single-photon emission computed tomography (SPECT). The radiolabels described above are useful in PET (e.g., ¹¹C, ¹⁸F or ⁷⁶Br) and SPECT (e.g., ¹²³I) imaging, with half-lives of about 20.4 minutes for ¹¹C, about 109 minutes for ¹⁸F, about 13 hours for ¹²³I, and about 16 hours for ⁷⁶Br. A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al.

The compounds can be administered using known techniques. See, for example, U.S. Pat. No. 5,969,144 to London et al., as noted. The compounds can be administered in formulation compositions that incorporate other ingredients, such as those types of ingredients that are useful in formulating a diagnostic composition. Compounds useful in accordance with carrying out the present invention most preferably are employed in forms of high purity. See, U.S. Pat. No. 5,853,696 to Elmalch et al.

After the compounds are administered to a subject (e.g., a human subject), the presence of that compound within the subject can be imaged and quantified by appropriate techniques in order to indicate the presence, quantity, and functionality. In addition to humans, the compounds can also be administered to animals, such as mice, rats, dogs, and monkeys. SPECT and PET imaging can be carried out using any appropriate technique and apparatus. See Villemagne et al., In: Arneric et al. (Eds.) Neuronal Nicotinic Receptors: Pharmacology and Therapeutic Opportunities, 235-250 (1998) and U.S. Pat. No. 5,853,696 to Elmalch et al., each herein incporated by reference, for a disclosure of representative imaging techniques.

### V. Biological Assays

Characterization of Interactions at Nicotinic Acetylcholine Receptors Materials and methods

Cell Lines.

SH-EP1-human α4β2 (Eaton et al., 2003), SH-EP1-human α4β4 (Gentry et al., 2003) and SH-EP1-α6β3β4α5 (Grinevich et al., 2005) cell lines were obtained from Dr. Ron Lukas (Barrow Neurological Institute). The SH-EP1 cell lines, PC12, SH-SY5Y and TE671/RD cells were maintained in proliferative growth phase in Dulbecco's modified Eagle's medium (Invitrogen, Carlsbad, Calif.) with 10% horse serum (Invitrogen), 5% fetal bovine serum (HyClone, Logan Utah), 1 mM sodium pyruvate, 4 mM L-glutamine. For maintenance of stable transfectants, the α4β2 and α4β4 cell media was supplemented with 0.25 mg/mL zeocin and 0.13 mg/mL hygromycin B. Selection was maintained for the α6β3β4α5 cells with 0.25 mg/mL of zeocin, 0.13 mg/mL of hygromycin B, 0.4 mg/mL of geneticin, and 0.2 mg/mL of blasticidin. HEK-human α7/RIC3 cells (obtained from J. Lindstrom, U. Pennsylvania) were maintained in proliferative growth phase in Dulbecco's modified Eagle's medium (Invitrogen) with 10% fetal bovine serum (HyClone, Logan, Utah), 1 mM sodium pyruvate, 4 mM L-glutamine, 0.6 mg/mL geneticin; 0.5 mg/ml zeocin. GH4C1-rat T6'S α7 cells recombinantly express the T6'S mutant rat α7 gene (Placzek et al., 2005). The T6'S mutant rat α7 gene construct in pClneo was obtained from Roger L. Papke (U. of Florida) and subcloned into pCEP4. The plasmid construct was transfected into GH4C1 cells, and a stable clone was isolated under hygromycin selection. The GH4C1-rat T6'S α7 cells were maintained in proliferative growth phase in Ham's F10 with L-glutamine, 10% horse serum, 5% fetal bovine serum and 0.15 mg/mL of hygromycin B.

### Receptor Binding Assays

Preparation of Membranes from Rat Tissues.

Rat cortices were obtained from Analytical Biological Services, Incorporated (ABS, Wilmington, Del.). Tissues were dissected from female Sprague-Dawley rats, frozen and shipped on dry ice. Tissues were stored at −20° C. until needed for membrane preparation. Cortices from 10 rats were pooled and homogenized by Polytron (Kinematica GmbH, Switzerland) in 10 volumes (weight:volume) of ice-cold preparative buffer (11 mM KCl, 6 mM KH2PO4, 137 mM NaCl, 8 mM Na2HPO4, 20 mM HEPES (free acid), 5 mM iodoacetamide, 1.5 mM EDTA, 0.1 mM PMSF pH 7.4). The resulting homogenate was centrifuged at 40,000 g for 20 minutes at 4° C. and the resulting pellet was re-suspended in 20 volumes of ice-cold water. After 60 minute incubation at 4° C., a new pellet was collected by centrifugation at 40,000 g for 20 minutes at 4° C. The final pellet was re-suspended in preparative buffer and stored at −20° C. On the day of the assay, tissue was thawed, centrifuged at 40,000 g for 20 minutes and then re-suspended in Dulbecco's Phosphate Buffered Saline, pH 7.4 (PBS, Invitrogen) to a final concentration of 2-3 mg protein/mL. Protein concentrations were determined using the Pierce BCA Protein Assay kit (Pierce Biotechnology, Rockford, Ill.), with bovine serum albumin as the standard.

Preparation of Membranes from Clonal Cell Lines.

Cells were harvested in ice-cold PBS, pH 7.4, then homogenized with a Polytron (Kinematica GmbH, Switzerland). Homogenates were centrifuged at 40,000 g for 20 minutes (4° C.). The pellet was re-suspended in PBS and protein concentration determined using the Pierce BCA Protein Assay kit (Pierce Biotechnology, Rockford, Ill.). Competition binding to receptors in membrane preparations. Binding to nicotinic receptors was assayed on membranes using standard methods adapted from published procedures (Lippiello and Fernandes 1986; Davies et al., 1999). In brief, membranes were reconstituted from frozen stocks and incubated for 2 h on ice in 150 μl assay buffer (PBS) in the presence of competitor compound (0.001 nM to 100 μM) and radioligand. [³H]-nicotine (L-(−)-[N-methyl-³H]-nicotine, 69.5 Ci/mmol, Perkin-Elmer Life Sciences, Waltham, Mass.) was used for human α4β2 binding studies. [³H]-epibatidine (52 Ci/mmol, Perkin-Elmer Life Sciences) was used for binding studies at the other nicotinic receptor subtypes. L-[Benzilic-4,4-³H] Quinuclidynyl Benzilate ([³H]QNB) was used for muscarinic receptor binding studies. Membrane source, radioligand and radioligand concentration for each receptor target are listed in Table 1. Incubation was terminated by rapid filtration on a multimanifold tissue harvester (Brandel, Gaithersburg, Md.) using GF/B filters presoaked in 0.33% polyethyleneimine (w/v) to reduce non-specific binding. Filters were washed 3 times with ice-cold PBS and the retained radioactivity was determined by liquid scintillation counting.

TABLE 2
Binding Parameters

| | | | Radioligand |
|---|---|---|---|
| | | | concentration |
| Binding target | Membrane Source | Radioligand | (nM) |
| Nicotinic, rat α4β2 | Rat cortex | [³H]epibatidine | 0.1 |
| Nicotinic, human α4β2 | SH-EP1-Human α4β2 cells | [³H]nicotine | 2 |
| Nicotinic, human α4β4 | SH-EP1-Human α4β4 cells | [³H]epibatidine | 0.25 |
| Nicotinic, human | SH-EP1-Human α6β3β4α5 | [³H]epibatidine | 0.5 |
| α6β3β4α5 | cells | | |
| Nicotinic, human α7 | HEK Human α7/RIC3 | [³H]epibatidine | 10 |
| Nicotinic, human α3β4α5 | SH-SY5Y cells | [³H]epibatidine | 1 |
| Nicotinic, human α1β1γδ | TE671/RD cells | [³H]epibatidine | 10 |
| Nicotinic, rat α3β4 | PC12 cells | [³H]epibatidine | 0.6 |
| Muscarinic, M1, M2 | Rat cortex | [³H]QNB | 2 |
| Muscarinic, M3 | TE671/RD cells | [³H]QNB | 1 |

Binding Data Analysis.

Binding data were expressed as percent total control binding. Replicates for each point were averaged and plotted against the log of drug concentration. The IC₅₀ (concentration of the compound that produces 50% inhibition of binding) was determined by least squares non-linear regression using GraphPad Prism software (GraphPAD, San Diego, Calif.). Ki was calculated using the Cheng-Prusoff equation (Cheng and Prusoff, 1973).

### Calcium Flux Functional Assays

Twenty-four to forty-eight hours prior to each experiment, cells were plated in 96 well black-walled, clear bottom plates (Corning, Corning, N.Y.) at 60-100,000 cells/well. On the day of the experiment, growth medium was gently removed, 200 μL 1×FLIPR Calcium 4 Assay reagent (Molecular Devices, Sunnyvale, Calif.) in assay buffer (20 mM HEPES, 7 mM TRIS base, 4 mM CaCl₂, 5 mM D-glucose, 0.8 mM MgSO₄, 5 mM KCl, 0.8 mM MgCl₂, 120 mM N-methyl D-glucamine, 20 mM NaCl, pH 7.4 for SH-EP1-human α4β2 cells or 10 mM HEPES, 2.5 mM CaCl₂, 5.6 mM D-glucose, 0.8 mM MgSO₄, 5.3 mM KCl, 138 mM NaCl, pH 7.4 with TRIS-base for all other cell lines) was added to each well and plates were incubated at 37° C. for 1 hour (29° C. for the 29° C.-treated SH-EP1-human α4β2 cells). For inhibition studies, competitor compound (10 pM-10 μM) was added at the time of dye addition. The plates were removed from the incubator and allowed to equilibrate to room temperature. Plates were transferred to a FLIPR Tetra fluorometric imaging plate reader (Molecular Devices) for addition of compound and monitoring of fluorescence (excitation 485 nm, emission 525 nm). The amount of calcium flux was compared to both a positive (nicotine) and negative control (buffer alone). The positive control was defined as 100% response and the results of the test compounds were expressed as a percentage of the positive control. For inhibition studies, the agonist nicotine was used at concentrations of 1 μM for SH-EP1-human α4β2 and SH-EP1-human α4β4 cells, 10 μM for PC12 and SH-SY5Y cells, and 100 μM for TE671/RD cells.

### Neurotransmitter Release

Dopamine release studies were performed using striatal synaptosomes obtained from rat brain as previously described (Bencherif et al., 1998). Striatal tissue from two rats (female, Sprague-Dawley, weighing 150-250 g) was pooled and homogenized in ice-cold 0.32 M sucrose (8 mL) containing 5 mM HEPES, pH 7.4, using a glass/glass homogenizer. The tissue was then centrifuged at 1,000×g for 10 minutes. The pellet was discarded and the supernatant was centrifuged at 12,500×g for 20 minutes. The resulting pellet was re-suspended in ice-cold perfusion buffer containing monoamine oxidase inhibitors (128 mM NaCl, 1.2 mM KH₂PO₄, 2.4 mM KCl, 3.2 mM CaCl₂, 1.2 mM MgSO₄, 25 mM HEPES, 1 mM ascorbic acid, 0.02 mM pargyline HCl and 10 mM glucose, pH 7.4) and centrifuged for 15 minutes at 23,000×g. The final pellet was re-suspended in perfusion buffer (2 mL) for immediate use.

The synaptosomal suspension was incubated for 10 minutes in a 37° C. shaking incubator to restore metabolic activity. [³H]Dopamine ([³H]DA, specific activity=28.0 Ci/mmol, NEN Research Products) was added at a final concentration of 0.1 μM and the suspension was incubated at 37° C. for another 10 minutes. Aliquots of perfusion buffer (100 μL) and tissue (100 μL) were loaded into the suprafusion chambers of a Brandel Suprafusion System (series 2500, Gaithersburg, Md.). Perfusion buffer (room temperature) was pumped into the chambers at a rate of approximately 0.6 mL/min for a wash period of 8 min. Competitor compound (10 pM-100 nM) was applied in the perfusion stream for 8 minutes. Nicotine (10 μM) was then applied in the perfusion stream for 48 seconds. Fractions (12 seconds each) were continuously collected from each chamber throughout the experiment to capture basal release and agonist-induced peak release and to re-establish the baseline after the agonist application. The perfusate was collected directly into scintillation vials, to which scintillation fluid was added. Released [³H]DA was quantified by scintillation counting. For each chamber, the integrated area of the peak was normalized to its baseline. Release was expressed as a percentage of release obtained with control nicotine in the absence of competitor. Within each assay, each test compound concentration was replicated using 2 chambers; replicates were averaged. The compound concentration resulting in half maximal inhibition (IC₅₀) of specific ion flux was defined.

### Patch Clamp Electrophysiology

Cell Handling.

After removal of GH4C1-rat T6'S α7 cells from the incubator, medium was aspirated, cells trypsinized for 3 minutes, gently triturated to detach them from the plate, washed twice with recording medium, and re-suspended in 2 ml of external solution (see below for composition). Cells were placed in the Dynaflow chip mount on the stage of an inverted Zeiss microscope (Carl Zeiss Inc., Thornwood, N.Y.). On average, 5 minutes was necessary before the whole-cell recording configuration was established. To avoid modification of the cell conditions, a single cell was recorded per single load. To evoke short responses, agonists were applied for 0.5 s using a Dynaflow system (Cellectricon, Inc., Gaithersburg, Md.), where each channel delivered pressure-driven solutions at either 50 or 150 psi.

Electrophysiology.

Conventional whole-cell current recordings were used. Glass microelectrodes (5-10 MΩ resistance) were used to form tight seals (>1 GΩ) on the cell surface until suction was applied to convert to conventional whole-cell recording. The cells were then voltage-clamped at holding potentials of −60 mV, and ion currents in response to application of ligands were measured. Whole-cell currents recorded with an Axon 700A amplifier were filtered at 1 kHz and sampled at 5 kHz by an ADC board 1440 (Molecular Devices). Whole-cell access resistance was less than 20 MΩ. Data acquisition of whole-cell currents was done using a Clampex 10 (Molecular Devices, Sunnyvale, Calif.), and the results were plotted using Prism 5.0 (GraphPad Software Inc., San Diego, Calif.). The experimental data are presented as the mean±S.E.M., and comparisons of different conditions were analyzed for statistical significance using Student's t and Two Way ANOVA tests. All experiments were performed at room temperature (22±1° C.). Concentration-response profiles were fit to the Hill equation and analyzed using Prism 5.0.

Solutions and Drug Application.

The standard external solution contained: 120 mM NaCl, 3 mM KCl, 2 mM MgCl₂, 2 mM CaCl₂, 25 mM D-glucose, and 10 mM HEPES and was adjusted to pH 7.4 with Tris base. Internal solution for whole-cell recordings consisted of: 110 mM Tris phosphate dibasic, 28 mM Tris base, 11 mM EGTA, 2 mM MgCl₂, 0.1 mM CaCl₂, and 4 mM Mg-ATP, pH 7.3. (Liu et al., 2008). To initiate whole-cell current responses, compounds were delivered by moving cells from the control solution to agonist-containing solution and back so that solution exchange occurred within ˜50 ms (based on 10-90% peak current rise times). Intervals between compound applications (0.5-1 min) were adjusted specifically to ensure the stability of receptor responsiveness (without functional rundown), and the selection of pipette solutions used in most of the studies described here was made with the same objective. (−)-Nicotine and acetylcholine (ACh), were purchased from Sigma-Aldrich (St. Louis, Mo.). All drugs were prepared daily from stock solutions.

To determine the inhibition of ACh induced currents by compounds of the present invention, we established a stable baseline recording applying 70 μM ACh (usually stable 5-10 consecutive applications). Then ACh (70 μM) was co-applied with test compound in a concentration range of 1 nM to 10 μM. Since tail of the current (current measured at the end of 0.5 s ACh application) underwent the most profound changes, inhibition and recovery plots represent amplitude of tail current.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A compound of Formula I: 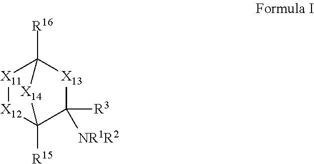
wherein
each of R¹ and R² individually is H, C₁₋₆ alkyl, or R¹ and R² combine with the nitrogen atom to which they are attached to form a 3- to 8-membered ring, which ring may be optionally substituted;
each of R¹⁵ and R¹⁶ individually is H, halogen, C₁₋₆ alkyl, C₁₋₆ haloalkyl, hydroxyl, C₁₋₆ alkoxy, or C₆₋₁₄ aryloxy;
R³ is H or C₁₋₆ alkyl;
each of X¹¹, X¹², X¹³, and X¹⁴ individually is —(CR⁴R⁵)—, where each of R⁴ and R⁵ is individually H, halogen, C₁₋₆ alkyl, C₁₋₆ haloalkyl, hydroxyl, C₁₋₆ alkoxy, or C₆₋₁₄ aryloxy;
or a pharmaceutically acceptable salt thereof.

2. A compound of Formula II: 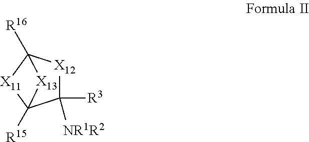
wherein
each of R¹ and R² individually is H, C₁₋₆ alkyl, or R¹ and R² combine with the nitrogen atom to which they are attached to form a 3- to 8-membered ring, which ring may be optionally substituted;
each of R¹⁵ and R¹⁶ individually is H, halogen, C₁₋₆ alkyl, C₁₋₆ haloalkyl, hydroxyl, C₁₋₆ alkoxy, or C₆₋₁₄ aryloxy;
R³ is H or C₁₋₆ alkyl;
each of X¹¹, X¹², and X¹³ individually is —(CR⁴R⁵)—, where each of R⁴ and R⁵ is individually is H, halogen, C₁₋₆ alkyl, C₁₋₆ haloalkyl, hydroxyl, C₁₋₆ alkoxy, or C₆₋₁₄ aryloxy;
or a pharmaceutically acceptable salt thereof.

3. A compound of Formula III: 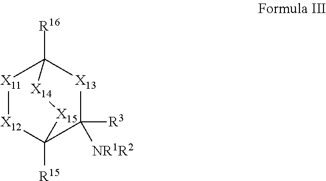
wherein
each of R¹ and R² individually is H, C₁₋₆ alkyl, or R¹ and R² combine with the nitrogen atom to which they are attached to form a 3- to 8-membered ring, which ring may be optionally substituted;
each of R¹⁵ and R¹⁶ individually is H, halogen, C₁₋₆ alkyl, C₁₋₆ haloalkyl, hydroxyl, C₁₋₆ alkoxy, or C₆₋₁₄ aryloxy;
R³ is H or C₁₋₆ alkyl;
each of X¹¹, X¹², X¹³, X¹⁴, and X¹⁵ individually is —(CR⁴R⁵)—, where each of R⁴ and R⁵ is individually is H, halogen, C₁₋₆ alkyl, C₁₋₆ haloalkyl, hydroxyl, C₁₋₆ alkoxy, or C₆₋₁₄ aryloxy;
or a pharmaceutically acceptable salt thereof.

4. The compound of claim 1, wherein optionally substituted is substitution with one or more C₁₋₆ alkyl, halogen, C₁₋₆ haloalkyl, C₁₋₆ alkoxy, or C₆₋₁₄ aryloxy.

5. The compound of claim 4, wherein R¹ is H and R² is C₁₋₆ alkyl.

6. The compound of claim 4, wherein R³ is C₁₋₆alkyl.

7. The compound of claim 4, wherein each of R¹ and R² are C₁₋₆ alkyl.

8. The compound of claim 7, wherein R³ is C₁₋₆ alkyl.

9. A pharmaceutical composition comprising a compound as claimed in claim 1 and a pharmaceutically acceptable carrier.

10. A method for the treatment or prevention of a disease or condition mediated by a neuronal nicotinic receptor comprising the administration of a compound as claimed in claim 1.

11. The method of claim 10, wherein the disease or condition is hypertension, nicotine addiction, depression, or anxiety.

12. (canceled)

13. (canceled)

14. (canceled)

15. (canceled)

16. (canceled)

17. A compound of Formulae IV, V, VI, or VII: 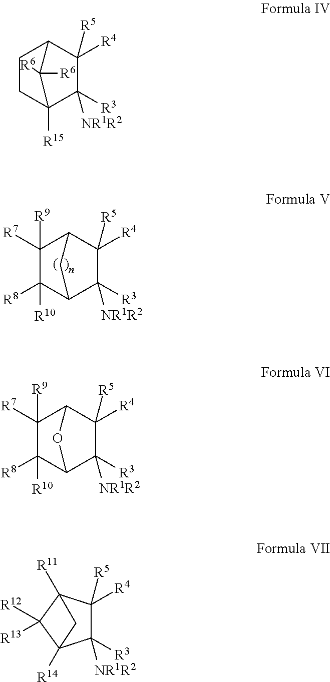
wherein
each of R¹ and R² individually is H, C₁₋₆ alkyl, or R¹ and R² combine with the nitrogen atom to which they are attached to form a 3- to 8-membered ring, which ring may be optionally substituted;
each of R³, R⁶, R¹¹, R¹², R¹³, and R¹⁴ is H or C₁₋₆ alkyl;
n is 1 or 2;
each of R⁴, R⁵, R⁷, R⁸, R⁹, and R¹⁰ individually is H, halogen, C₁₋₆ alkyl, C₁₋₆ haloalkyl, hydroxyl, C₁₋₆ alkoxy, or C₆₋₁₄ aryloxy;
R¹⁵ is H or methyl;
or a pharmaceutically acceptable salt thereof.

18. The compound of claim 17, wherein optionally substituted is substitution with one or more C₁₋₆ alkyl, halogen, C₁₋₆ haloalkyl, C₁₋₆ alkoxy, or C₆₋₁₄ aryloxy.

19. The compound of claim 18, wherein R¹ is H and R² is C₁₋₆ alkyl.

20. The compound of claim 18, wherein R³ is C₁₋₆ alkyl.

21. The compound of claim 18, wherein each of R¹ and R² are C₁₋₆ alkyl.

22. The compound of claim 21, wherein R³ is C₁₋₆ alkyl.

23. A pharmaceutical composition comprising a compound as claimed in claim 17 and a pharmaceutically acceptable carrier.

24. A method for the treatment or prevention of a disease or condition mediated by a neuronal nicotinic receptor comprising the administration of a compound as claimed in claim 17.

25. The method of claim 24, wherein the disease or condition is hypertension, nicotine addiction, depression, or anxiety.

26. (canceled)

27. (canceled)

28. (canceled)

29. (canceled)

30. (canceled)

31. The compound of claim 2, wherein optionally substituted is substitution with one or more C₁₋₆ alkyl, halogen, C₁₋₆ haloalkyl, C₁₋₆ alkoxy, or C₆₋₁₄ aryloxy.

32. The compound of claim 31, wherein R¹ is H and R² is C₁₋₆ alkyl.

33. The compound of claim 31, wherein R³ is C₁₋₆ alkyl.

34. The compound of claim 31, wherein each of R¹ and R² are C₁₋₆ alkyl.

35. The compound of claim 34, wherein R³ is C₁₋₆ alkyl.

36. A pharmaceutical composition comprising a compound as claimed in claim 2 and a pharmaceutically acceptable carrier.

37. A method for the treatment or prevention of a disease or condition mediated by a neuronal nicotinic receptor comprising the administration of a compound as claimed in claim 2.

38. The method of claim 37, wherein the disease or condition is hypertension, nicotine addiction, depression, or anxiety.

39. The compound of claim 3, wherein optionally substituted is substitution with one or more C₁₋₆ alkyl, halogen, C₁₋₆ haloalkyl, C₁₋₆ alkoxy, or C₆₋₁₄ aryloxy.

40. The compound of claim 39, wherein R¹ is H and R² is C₁₋₆ alkyl.

41. The compound of claim 39, wherein R³ is C₁₋₆ alkyl.

42. The compound of claim 39, wherein each of R¹ and R² are C₁₋₆ alkyl.

43. The compound of claim 42, wherein R³ is C₁₋₆ alkyl.

44. A pharmaceutical composition comprising a compound as claimed in claim 3 and a pharmaceutically acceptable carrier.

45. A method for the treatment or prevention of a disease or condition mediated by a neuronal nicotinic receptor comprising the administration of a compound as claimed in claim 3.

46. The method of claim 45, wherein the disease or condition is hypertension, nicotine addiction, depression, or anxiety.
